SEQUENCE LISTING (2 sequences)

SEQ ID NO: 1 - DNA, 18 bp, synthetic construct
  source 1..18: mol_type = other DNA
ccatcccaggccgactaa

SEQ ID NO: 2 - DNA, 23 bp, synthetic construct
  source 1..23: mol_type = other DNA
aatttcagagcattggccataga

(19) United States
(12) Patent Application Publication
(10) Pub. No.: US 2026/0109926 A1
(43) Pub. Date: Apr. 23, 2026

(54) PRODUCTION METHOD OF CELLS HAVING EXOGENOUS MITOCHONDRIA INTRODUCED THEREINTO

(71) Applicant: TOKYO UNIVERSITY OF SCIENCE FOUNDATION, Tokyo (JP)

(72) Inventors: Kosuke KUSAMORI, Tokyo (JP); Makiya NISHIKAWA, Tokyo (JP); Kaito ANDO, Tokyo (JP); Yoshikazu HIGAMI, Tokyo (JP); Masaki KOBAYASHI, Tokyo (JP); Mai KANAI, Tokyo (JP); Taisei YOKOYAMA, Tokyo (JP)

(21) Appl. No.: 19/120,533

(22) PCT Filed: Oct. 23, 2023
(86) PCT No.: PCT/JP2023/038203

§ 371 Date: Apr. 11, 2025

(30) Foreign Application Priority Data
Oct. 26, 2022 (JP) 2022-171254
Jan. 25, 2023 (JP) 2023-009196

(51) Int. Cl. C12M 1/00

(52) U.S. Cl. CPC C12M 23/20

(57) ABSTRACT

A method of producing cells into which exogenous mitochondria have been introduced, the production method including culturing recipient cells using cell culture equipment in which mitochondria isolated from donor cells are coated on the culture surface, and incorporating the mitochondria into the recipient cells. Cells produced by the method, the cells having exogenous mitochondria introduced thereinto; a cell culture substrate usable in the aforesaid production method and a method for manufacturing the same; and a cell culture kit.

### TECHNICAL FIELD

The present invention relates to a production method of cells having exogenous mitochondria introduced thereinto.

### BACKGROUND ART

Mitochondria are one of cell organelles existing in eukaryotic cells, and play an important role in adenosine triphosphate (ATP) production and apoptosis. In recent years, it has been reported that, for example, the function of cells is raised and the effect of cell transplantation therapy is increased by introducing exogenous mitochondria into cells for transplantation (see e.g. Non-Patent Document 1), and various methods for introducing exogenous mitochondria into cells have been investigated.

Known methods for introducing exogenous mitochondria into cells include, (1) a method in which exogenous mitochondria are incubated with recipient cells (see e.g. Patent Document 1); (2) a method in which the surface of exogenous mitochondria is modified with a cell-penetrating peptide and then incubated with recipient cells (see e.g. Non-Patent Document 2); and (3) a method in which exogenous mitochondria are introduced into recipient cells by microinjection (see e.g. Patent Document 2), and the like.

The method in (1) above, however, has a problem in that the operation is easy, but at the same time the surface of mitochondria is negatively charged and thus introduction efficiency is low. The method in (2) above has higher introduction efficiency than in the method in (1) above, but has concerns that time and effort are required for the operation to modify mitochondria, and moreover cells are damaged due to the cell-penetrating peptide, a cationic substance. In the method in (3) above, mitochondria can be certainly introduced into recipient cells; however, an advanced technique is required for the operation therefor, and moreover the number of recipient cells, the object, is limited. Furthermore, the method in (3) above has concerns about damages to cells.

### CITATION LIST

### Patent Document

Patent Document 1: Japanese Unexamined Patent Application (Translation of PCT Application), Publication No. 2021-532095
Patent Document 2: Japanese Unexamined Patent Application (Translation of PCT Application), Publication No. 2019-500395

### Non-Patent Document

Non-Patent Document 1: Guo, Y. et al., Stem Cell Res. Ther., 11, 245, 2020
Non-Patent Document 2: Maeda, H. et al., J. Cell. Mol. Med., 24, 5007-5020, 2020

### DISCLOSURE OF THE INVENTION

### Problems to be Solved by the Invention

Therefore, a subject of the present invention is to provide a novel production method of cells having exogenous mitochondria introduced thereinto.

### Means for Solving the Problems

A specific means to solve the above subject includes the following embodiments.

<1> A production method of cells having exogenous mitochondria introduced thereinto, the production method including, culturing recipient cells using cell culture equipment in which the culture surface is coated with mitochondria isolated from donor cells to make the recipient cells incorporate the mitochondria.

<2> The production method described in <1>, wherein the cell culture equipment is a cell culture vessel or a cell culture carrier.

<3> Cells having exogenous mitochondria introduced thereinto, produced by the production method described in <1> or <2>.

<4> The cells described in <3>, wherein the protein amount in the introduced exogenous mitochondria is 0.6 μg or more per 2.0×10⁵ cells.

<5> Cell culture equipment, including the culture surface coated with isolated mitochondria.

<6> The cell culture equipment described in <5>, which is a cell culture vessel or a cell culture carrier.

<7> A production method of a cell culture vessel, including a culture surface coated with isolated mitochondria, the production method including,
centrifuging the cell culture vessel with a liquid containing the isolated mitochondria added to the cell culture vessel to attach the mitochondria to the culture surface of the cell culture vessel.

<8> A cell culture kit, including a cell culture equipment and a liquid containing isolated mitochondria.

### Effects of the Invention

According to the present invention, it is possible to provide a novel production method of cells having exogenous mitochondria introduced thereinto.

### BRIEF DESCRIPTION OF THE DRAWINGS

FIG. 1 is images showing mitochondria attached to the inside bottom surface of a dish when adding green fluorescent protein (GFP)-labeled mitochondria to the dish and incubating them for a predetermined time.

FIG. 2A is a graph showing, when centrifuging a suspension obtained by suspending GFP-labeled mitochondria in phosphate buffered saline (PBS) at various centrifugal forces for 10 minutes, the fluorescence intensity of the supernatants.

FIG. 2B is a graph showing, when centrifuging a suspension obtained by suspending GFP-labeled mitochondria in PBS at various centrifugal forces for 10 minutes and suspending the obtained mitochondrial precipitate (pellet) in PBS again, the fluorescence intensity of the pellet suspensions.

FIG. 3 is images showing, when adding mitochondria stained with carboxy fluorescein succinimidyl ester (CSFE) to a plate and then carrying out plate centrifugation and when not carrying out plate centrifugation, mitochondria attached to the inside bottom surface of wells.

FIG. 4 is a graph showing, when using a plate having the inside bottom surface of wells coated with mitochondria by adding GFP-labeled mitochondria to the plate, carrying out plate centrifugation, and then incubating them for a predetermined time, and a plate coated with mitochondria only by incubation without plate centrifugation, C3H10T1/2 cells were cultured and the cultured cells were analyzed by flow cytometry, the geometric mean fluorescence intensity (gMFI) of the cells.

FIG. 5 is confocal laser microscope images showing, when C3H10T1/2 cells were seeded on a plate coated with GFP-labeled mitochondria and when C3H10T1/2 cells were seeded on a plate not coated with mitochondria and mitochondria were then added to the attached C3H10T1/2 cells, mitochondria incorporated into the cells.

FIG. 6 is a graph showing, when about a case where C3H10T1/2 cells were seeded on a plate coated with GFP-labeled mitochondria and a case where C3H10T1/2 cells were seeded on a plate not coated with mitochondria and mitochondria were then added to the attached C3H10T1/2 cells, C3H10T1/2 cells after culturing were analyzed by flow cytometry, the geometric mean fluorescence intensity of the cells.

FIG. 7 is a graph showing, when C3H10T1/2 cells were seeded on a plate coated with mitochondria and when C3H10T1/2 cells were seeded on a plate not coated with mitochondria and mitochondria were then added to the attached C3H10T1/2 cells, the cell number (relative value) after culturing.

FIG. 8 is a graph showing, when C3H10T1/2 cells were seeded on a plate coated with mitochondria, the amount of ATP production in C3H10T1/2 cells.

FIG. 9 is a graph showing, when C3H10T1/2 cells were cultured on a plate coated with mitochondria at various numbers, the cell number (relative value) after culturing.

FIG. 10 is a graph showing, when oligomycin-treated C3H10T1/2 cells were cultured on a plate coated with mitochondria at various numbers, the cell number (relative value) after culturing.

FIG. 11A is images showing in vivo images when mitochondria-introduced NanoLuc luciferase-expressing C3H10T1/2 cells or non-treated NanoLuc luciferase-expressing C3H10T1/2 cells were subcutaneously administered to the back of mice.

FIG. 11B is a graph showing the plasma luciferase activity when mitochondria-introduced NanoLuc luciferase-expressing C3H10T1/2 cells or non-treated NanoLuc luciferase-expressing C3H10T1/2 cells were subcutaneously administered to the back of mice.

FIG. 12A is a graph showing the serum AST value when mitochondria-introduced NanoLuc luciferase-expressing C3H10T1/2 cells or non-treated NanoLuc luciferase-expressing C3H10T1/2 cells were intravenously administered to carbon tetrachloride (CC14)-induced hepatopathy model mice.

FIG. 12B is a graph showing the serum ALT value when mitochondria-introduced NanoLuc luciferase-expressing C3H10T1/2 cells or non-treated NanoLuc luciferase-expressing C3H10T1/2 cells were intravenously administered to CC14-induced hepatopathy model mice.

FIG. 13 is a graph showing, when various cells (C3H10T1/2 cells, Hepa1-6 cells, HEK293 cells, HaCaT cells) were seeded on a plate coated with mitochondria, and when various cells were seeded on a plate not coated with mitochondria, the cell number (relative value) after culturing.

FIG. 14A is a graph showing, when HEK293 cells were seeded on a plate coated with mitochondria and when HEK293 cells were seeded on a plate and mitochondria were then added thereto, the protein amount in mitochondria incorporated per 2.0×10⁵ cells.

FIG. 14B is a graph showing, when HaCaT cells were seeded on a plate coated with mitochondria and when HaCaT cells were seeded on a plate and mitochondria were then added thereto, the protein amount in mitochondria incorporated per 2.0×10⁵ cells.

FIG. 15 is a graph showing, when C3H10T1/2 cells were cultured in the presence of polyethylenimine (PEI) at various concentrations, the cell number (relative value) after culturing.

### PREFERRED MODE FOR CARRYING OUT THE INVENTION

The production method of cells having exogenous mitochondria introduced thereinto according to the present embodiment (hereinafter simply referred to as “production method”) includes culturing recipient cells using cell culture equipment in which the culture surface is coated with mitochondria isolated from donor cells to make the recipient cells incorporate the mitochondria (exogenous mitochondria).

The “donor cells” mean cells which provide mitochondria, and the “recipient cells” mean cells into which mitochondria are introduced. The donor cells and the recipient cells may be cells including normal mitochondria or cells including dysfunctional mitochondria in which mitochondrial DNA is mutated. When the recipient cells are cells including dysfunctional mitochondria, at least a part of endogenous mitochondria may be removed in advance by a conventionally known means.

The biological species from which the donor cells and the recipient cells are derived is not particularly restricted as long as mitochondria are contained in the cells thereof, and may be an animal or a plant. Examples of the biological species include mammals such as mouse, rat, dog, sheep, monkey and human. The donor cells and the recipient cells may be derived from different individuals in the same biological species or derived from different biological species.

The cell type of the donor cells and the recipient cells is not particularly restricted. When the donor cells and the recipient cells are animal cells, examples of the cell type include muscle cells, hepatic cells, fibroblasts, epithelium cells, nerve cells, fat cells and mesenchymal stem cells. The donor cells and the recipient cells may be cells from the same type or cells from different types.

An optional method can be adopted as the method for isolating mitochondria from donor cells, and a commercially available kit can be used as needed. Known methods for isolating mitochondria from donor cells include, a method in which after crushing donor cells the mitochondrial fraction is isolated by centrifugation, a method in which after forming a hole on the cell membranes of donor cells, the mitochondrial fraction is isolated by centrifugation, and the like. Among these, the method in which after forming a hole on the cell membranes of donor cells, the mitochondrial fraction is isolated is preferred from the viewpoint of isolating mitochondria with less damage, and particularly a method in which after forming a hole on the cell membranes of donor cells using SLO, the mitochondrial fraction is isolated (see e.g. Shibata, T. et al., Biochem. Biophys. Res. Commun., 463, 563-568, 2015) is preferred.

As the cell culture equipment which is coated with the isolated mitochondria, conventionally known cell culture equipment which can culture recipient cells can be used. Examples of the cell culture equipment include cell culture vessels such as a dish, a plate and a flask; cell culture carriers such as a micro carrier; and the like. Examples of the material for the cell culture equipment include glass; synthetic polymers such as polyethylene, polypropylene and polystyrene; natural polymers such as cellulose and collagen; metal; and the like. The cell culture equipment may be also a cell culture vessel for producing a cell sheet, in which a temperature-responsive polymer is immobilized on the culture surface thereof. Examples of the temperature-responsive polymer include poly(N-isopropylacrylamide) and the like.

The culture surface of the cell culture equipment is coated with the isolated mitochondria. The “culture surface” is only needed to be a surface which can come into contact with recipient cells at the time of culturing, and can be the inside bottom surface in the cell culture vessel and the outer surface in the cell culture carrier.

As a method for coating the culture surface of the cell culture equipment with the isolated mitochondria, any method can be adopted as long as the mitochondria are attached to the culture surface. The mitochondria can be commonly attached to the culture surface only by allowing a liquid containing the isolated mitochondria to stand with the liquid brought into contact with the culture surface. The time to stand is preferably 12 hours or longer and more preferably 24 hours or longer. The liquid to suspend the isolated mitochondria is not particularly restricted, and may be a medium for cell culturing or a buffer such as PBS.

When the cell culture equipment is a cell culture vessel such as a dish or a plate, by centrifuging the cell culture vessel with a liquid containing the isolated mitochondria added to the cell culture vessel, the mitochondria can be more efficiently and certainly attached to the culture surface. The centrifugation conditions are not particularly restricted and can be properly selected, and examples thereof include 30 seconds to 60 minutes in 300 g to 3200 g. It should be noted that after centrifugation, the cell culture vessel may be further allowed to stand with the liquid containing the isolated mitochondria added thereto.

The number of mitochondria coated on the culture surface of cell culture equipment is properly set depending on the types of cell culture equipment and the number of recipient cells. As an example, the culture surface of the cell culture equipment is preferably coated with mitochondria isolated from donor cells twice to 100 times the number of recipient cells. The density of mitochondria coated on the culture surface of the cell culture equipment is, for example, preferably 5 to 15 μg/cm².

After obtaining the cell culture equipment having the culture surface coated with the isolated mitochondria as described above, the mitochondria (exogenous mitochondria) can be incorporated into recipient cells by culturing recipient cells using the cell culture equipment. When culturing recipient cells, cell culture equipment coated in advance with the isolated mitochondria may be used. Alternatively, when culturing recipient cells, the culture surface of cell culture equipment may be coated with the isolated mitochondria using a cell culture kit including a cell culture equipment and a liquid containing the isolated mitochondria.

The cell concentration when culturing recipient cells is not particularly restricted. An example of the cell concentration is 1.0×10⁴ cells/mL to 5.0×10⁵ cells/mL.

Various media are used to culture recipient cells depending on the types of recipient cells. In addition, usual cell culture conditions can be adopted as conditions of culturing recipient cells. For example, the cells are cultured at a temperature of 30 to 40° C., a relative humidity of 90 to 98%, and a CO₂ concentration of 3 to 7%. The culture time is, for example, preferably 3 to 72 hours and more preferably 6 to 24 hours.

Mitochondria in cells can be enriched and the cell function, proliferative properties, etc., of cells can be raised by introducing exogenous mitochondria into recipient cells as described above. In addition, the cell function, proliferative properties, etc., can be improved by introducing normal exogenous mitochondria into recipient cells in which at least a part of dysfunctional endogenous mitochondria is removed.

According to the production method according to the present embodiment particularly, a much higher amount of exogenous mitochondria can be introduced into cells than in the method in which exogenous mitochondria are incubated with recipient cells (see e.g. Patent Document 1). The protein amount in exogenous mitochondria introduced into cells by the production method according to the present embodiment is, for example, 0.6 μg or more per 2.0×10⁵ cells, preferably 0.8 μg or more and more preferably 1.0 μg or more. In the production method according to the present embodiment, there is less damage to cells than in the method in which a cell-penetrating peptide, a cationic substance, is utilized (see e.g. Non-Patent Document 2) and the method in which microinjection is utilized (see e.g. Patent Document 2).

Cells having exogenous mitochondria introduced thereinto can be suitably used for e.g. cell medicine and material production. The cells having exogenous mitochondria introduced thereinto tend to have longer survival duration after transplantation, and thus the effect of cell transplantation therapy can be increased and material production using cells can be optimized. A cell sheet may be also produced using the cells having exogenous mitochondria introduced thereinto. The obtained cell sheet can be suitably used for regenerative medicine etc. It should be noted that a cell sheet having exogenous mitochondria introduced thereinto can be also directly obtained by coating a cell culture vessel for producing a cell sheet, in which a temperature-responsive polymer is immobilized on the culture surface, with mitochondria and then culturing recipient cells in the cell culture vessel.

### EXAMPLES

The present invention will now be described in more detail by way of Examples thereof. It should be noted, however, that the present invention is not restricted to these Examples.

### Preparation Example 1: The Isolation of Mitochondria (SLO Method)

The following two types of cells were prepared as donor cells to isolate mitochondria (hereinafter referred to as “mt”).

### C3H10T1/2 Cells: Mouse Mesenchymal Stem Cells

3T3-L1-mt-GFP cells: mouse fibroblast cell line expressing a fused protein of mitochondria transfer cox8a signal and GFP

First, C3H10T1/2 cells or 3T3-L1-mt-GFP cells were seeded on a 15 cm dish (Thermo Fisher Scientific) at a cell number of 1.0×10⁶ cells and cultured for 3 days. After culturing, the cells were washed with PBS, and the cells were recovered using a trypsin/EDTA solution (2.5 g/L trypsin, 1 mmol/L EDTA). The recovered cells were suspended in a HEPES-CH₃COOK buffer (480 μL) to which 1 μg/mL streptolysin O (FUJIFILM Wako Pure Chemical Corporation) (20 μL) had been added, and the obtained suspension was incubated at room temperature for 1 to 5 minutes and then allowed to stand on ice for 10 minutes. Subsequently, the cells were washed with a 4° C. Tris-sucrose buffer and then suspended in a Tris-sucrose buffer, and the obtained suspension was incubated at 37° C. for 10 minutes. The cell suspension was pipetted 200 times and centrifuged (400 g, 10 minutes, 4° C.) to collect the supernatant containing mitochondria. The collected supernatant was further centrifuged (7000 g, 10 minutes, 4° C.) to remove the supernatant. The obtained mitochondrial fraction was suspended in a 15% FBS-containing DMEM medium etc., and the obtained suspension was stored at 4° C. The operation to isolate mitochondria using streptolysin 0 as described above will now be referred to as “SLO method”.

### Test Example 1: The Attachment of Mitochondria to the Inside Bottom Surface of a Dish

First, mitochondria were isolated from 1.6×10⁷ cells of 3T3-L1-mt-GFP cells using the SLO method. Mitochondrial pellets were suspended in a 10% FBS-containing DMEM medium (530 μL), and the obtained suspension was added to a 35 mm glass bottom dish coated with polylysine so that mitochondria were 3.0×10⁶ cells mt/well, and incubated for predetermined times (1, 12 and 24 hours). Subsequently, mitochondria were immobilized using a 4% paraformaldehyde phosphate buffer, and a mounting agent containing DAPI (4′,6-diamidino-2-phenylindole) (Mounting Medium with DAPI, Vector laboratories) was added thereto. Then fluorescence images were taken using a confocal laser microscope (SP8, Leica) and an imaging software (LAS X Life Science, Leica).

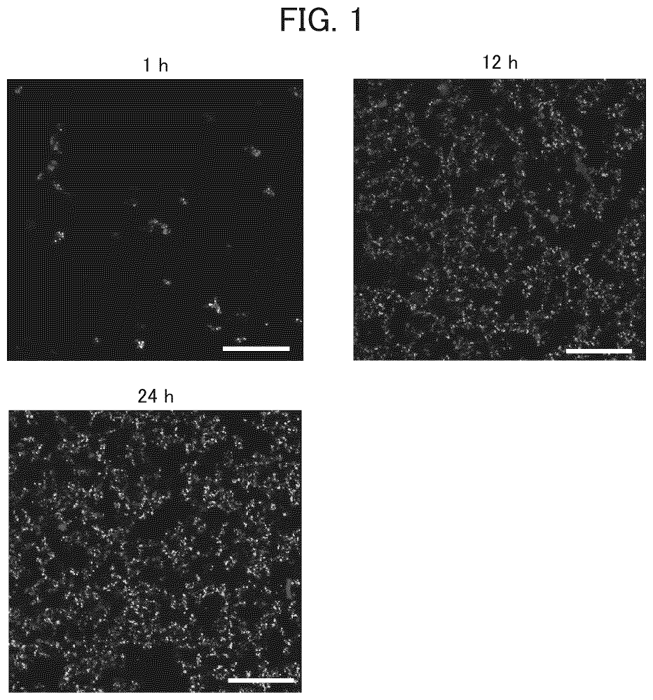

The fluorescence images are shown in FIG. 1 (scale bar: 40 μm). As can be seen from FIG. 1, mitochondria were observed to be attached to the inside bottom surface of the dish by incubation for about 12 hours.

### Test Example 2: The Optimization of Centrifugation Conditions for Coating with Mitochondria

First, mitochondria were isolated from 2.0×10⁷ cells of 3T3-L1-mt-GFP cells using the SLO method. The isolated mitochondria were suspended in PBS at 3.0×10⁶ cells mt/300 μL, and the obtained suspension was centrifuged at each centrifugal force (300 g, 500 g, 1000 g, 1500 g, 2000 g). The temperature and the time at the time of centrifugation are maintained to 4° C. and 10 minutes, respectively. The supernatant (300 μL) after centrifugation was collected, and mitochondrial precipitate (pellet) was suspended in PBS (300 μL) again. Then the supernatant and the pellet suspension were each added to a 96 well plate (Corning) at 90 μL/well, and the fluorescence intensity was measured using a microplate reader (ARVO-MX, PerkinElmer).

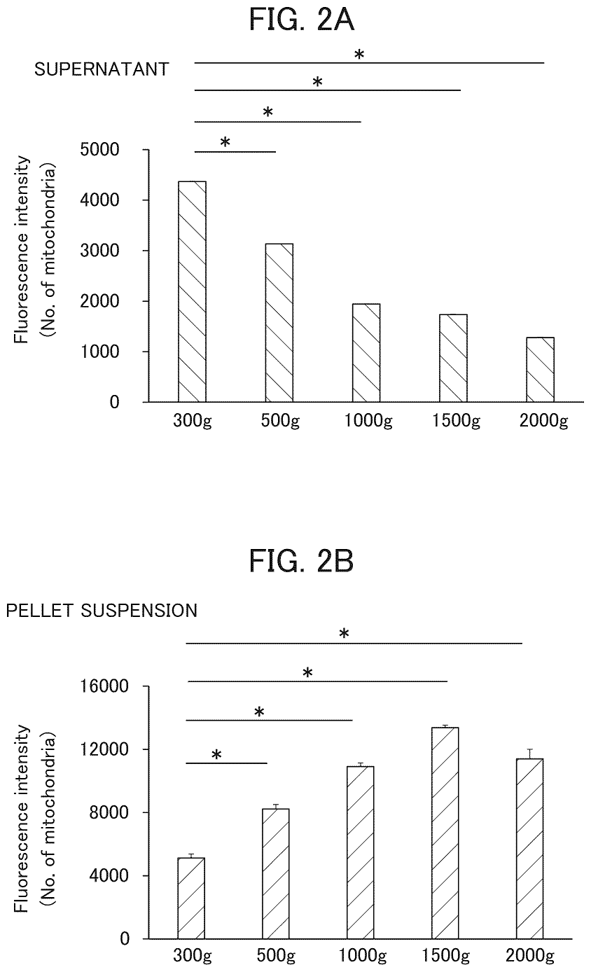

The fluorescence intensity of the supernatant and the fluorescence intensity of the pellet suspension are shown in FIG. 2A, and FIG. 2B, respectively. Each data in graphs shows the mean value±standard deviation of 3 samples. In addition, the “*” in the graphs indicates that there is a statistical significance (*p<0.05; Dunnett's test). As can be seen from FIG. 2A and FIG. 2B, when the centrifugal force was 1500 g or more, the fluorescence intensity of the supernatant was further reduced while the fluorescence intensity of the pellet suspension was further increased, and particularly preferred results were obtained.

### Test Example 3: Coating with Mitochondria by Plate Centrifugation

First, mitochondria were isolated from 2.0×10⁷ cells of C3H10T1/2 cells using the SLO method. A 10 μM CFSE solution (1 mL) was added to the isolated mitochondria, and the obtained mixture was allowed to stand on ice for 30 minutes to stain the mitochondria. After washing with PBS twice, the stained mitochondria were added to a 12 well plate at 3.0×10⁶ cells mt/well, and plate centrifugation (1500 g, 10 minutes, 4° C.) was carried out. Then fluorescence images were taken using a digital fluorescence microscope (BZ-9000, KEYENCE CORPORATION).

For comparison, a group of the same operation as above except for without plate centrifugation, and a group without the addition of mitochondria and plate centrifugation were also prepared, and fluorescence images were taken in the same manner as above.

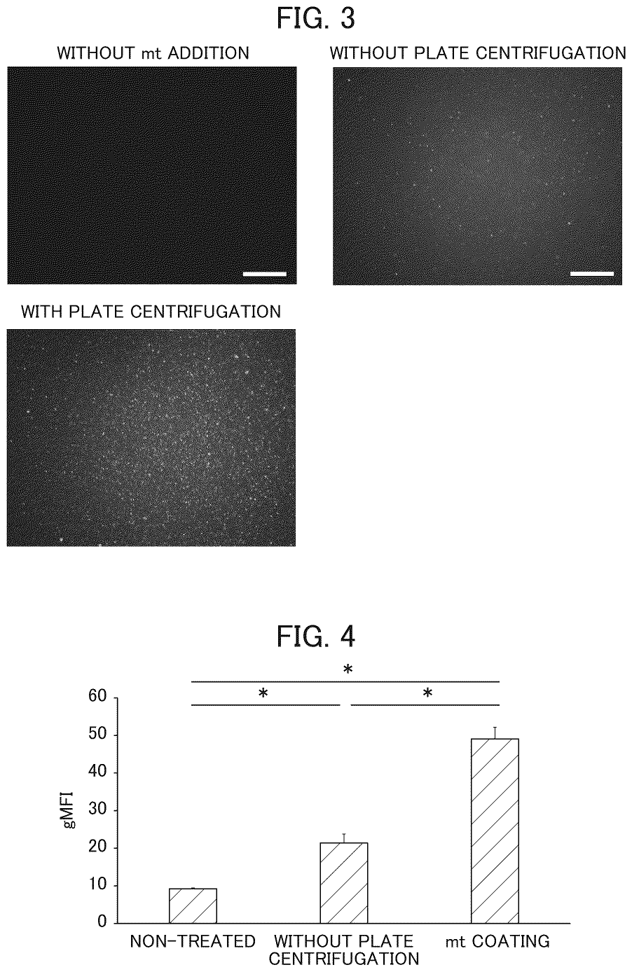

The fluorescence images are shown in FIG. 3 (scale bar: 500 μm). As can be seen from FIG. 3, the inside bottom surface of the wells were observed to be coated with mitochondria by plate centrifugation for 10 minutes. The operation to coat the inside bottom surface of the wells with mitochondria by plate centrifugation as described above will now be referred to as “mt coating”.

### Test Example 4: The Evaluation of Incorporation of Mitochondria (FACS)

First, mitochondria were isolated from 2.0×10⁷ cells of 3T3-L1-mt-GFP cells using the SLO method. The mitochondria were added to a 12 well plate at 3.0×10⁶ cells mt/well, and plate centrifugation (1500 g, 10 minutes, 4° C.) was carried out, followed by incubation for 12 hours to coat the inside bottom surface of the wells with the mitochondria. Subsequently, C3H10T1/2 cells were seeded at 3.0×10⁵ cells/well and cultured for 24 hours. After culturing, the cells were detached from the wells using a trypsin/EDTA solution (2.5 g/L trypsin, 1 mmol/L EDTA) and suspended in PBS. Then the cells in the suspended state were immobilized using a 4% paraformaldehyde phosphate buffer, which was replaced with PBS, and the incorporation of mitochondria was then analyzed using a flow cytometer (BD FACS Lyric, Beckton Dickinson). An analytical software (FlowJo software version 8.7, Beckton Dickinson) was used for data analysis.

For comparison, a group of the same operation as above except for without plate centrifugation, and a non-treated group of the same operation as above except for using a 12 well plate not coated with mitochondria were also prepared, and analyzed using a flow cytometer in the same manner as above.

The geometric mean fluorescence intensity in each group is shown in FIG. 4. Each data in the graph shows the mean value±standard deviation of 3 samples. In addition, the “*” in the graph indicates that there is a statistical significance (*p<0.05; Tukey-Kramer's test). As can be seen from FIG. 4, mitochondria were incorporated also when mitochondria were coated only by incubation for 12 hours; however, the incorporation of mitochondria was significantly promoted by plate centrifugation.

### Test Example 5: The Evaluation of Incorporation of Mitochondria (Microscope Observation)

First, mitochondria were isolated from 2.0×10⁷ cells of 3T3-L1-mt-GFP cells using the SLO method. The mitochondria were added to a 12 well plate at 3.0×10⁶ cells mt/well, and plate centrifugation (1500 g, 10 minutes, 4° C.) was carried out to coat the inside bottom surface of the wells with the mitochondria. Subsequently, C3H10T1/2 cells were seeded at 3.0×10⁵ cells/well and cultured for 24 hours. After culturing, the cells were detached from the wells using a trypsin/EDTA solution (2.5 g/L trypsin, 1 mmol/L EDTA), were seeded on a 35 mm glass bottom dish and cultured for 12 hours. Subsequently, the cells in the attached state were immobilized using a 4% paraformaldehyde phosphate buffer, and a mounting agent containing DAPI (Mounting Medium with DAPI, Vector laboratories) was added thereto. Then fluorescence images were taken using a confocal laser microscope (SP8, Leica) and an imaging software (LAS X Life Science, Leica).

For comparison, a group of not coating the inside bottom surface of the wells with mitochondria and adding mitochondria to the attached cells, and a non-treated group of the same operation as above except for using a 12 well plate not coated with mitochondria were also prepared.

In the group of adding mitochondria to the attached cells, C3H10T1/2 cells were first seeded on a 12 well plate so that the cell density when adding mitochondria was 3.0×10⁵ cells/well, and cultured until the next day. Mitochondria were isolated from 2.0×10⁷ cells of 3T3-L1-mt-GFP cells using the SLO method, and the isolated mitochondria were added at 3.0×10⁶ cells mt/well and cultured for 24 hours. After culturing, the cells were detached from the wells using a trypsin/EDTA solution (2.5 g/L trypsin, 1 mmol/L EDTA), seeded on a 35 mm glass bottom dish and cultured for 12 hours. Then the cells in the attached state were immobilized using a 4% paraformaldehyde phosphate buffer, and fluorescence images were taken in the same manner as above.

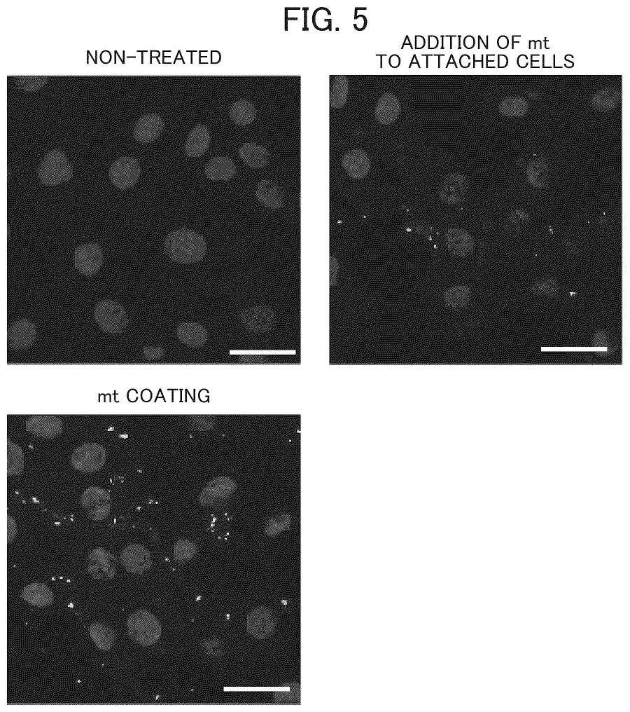

The fluorescence images are shown in FIG. 5 (scale bar: 40 μm). As can be seen from FIG. 5, when culturing cells on a plate having the inside bottom surface of the wells coated with mitochondria, the amount of the incorporated mitochondria was increased compared to when adding mitochondria to the attached cells without coating the inside bottom surface of the wells with mitochondria.

### Test Example 6: The Comparison of the Amount of Incorporated Mitochondria (FACS)

First, mitochondria were isolated from 3.0×10⁷ cells of 3T3-L1-mt-GFP cells using the SLO method. The mitochondria were added to a 12 well plate at 3.0×10⁶ cells mt/well, and plate centrifugation (1500 g, 10 minutes, 4° C.) was carried out to coat the inside bottom surface of the wells with the mitochondria. Subsequently, C3H10T1/2 cells were seeded at 3.0×10⁵ cells/well and cultured for 24 hours. After culturing, the cells were detached from the wells using a trypsin/EDTA solution (2.5 g/L trypsin, 1 mmol/L EDTA) and suspended in PBS. Then the cells in the suspended state were immobilized using a 4% paraformaldehyde phosphate buffer, which was replaced with PBS, and then the incorporation of mitochondria was analyzed using a flow cytometer (BD FACS Lyric, Beckton Dickinson). An analytical software (FlowJo software version 8.7, Beckton Dickinson) was used for data analysis.

For comparison, a group of not coating the inside bottom surface of the wells with mitochondria and adding mitochondria to the attached cells, and a non-treated group of using a 12 well plate not coated with mitochondria were also prepared.

In the group of adding mitochondria to the attached cells, C3H10T1/2 cells were first seeded on a 12 well plate so that the cell density when adding mitochondria was 3.0×10⁵ cells/well, and cultured until the next day. Mitochondria were isolated from 3.0×10⁷ cells of 3T3-L1-mt-GFP cells using the SLO method, and the isolated mitochondria were added at 3.0×10⁶ cells mt/well and cultured for 24 hours. After culturing, the cells were detached from the wells using a trypsin/EDTA solution (2.5 g/L trypsin, 1 mmol/L EDTA) and suspended in PBS. Then the cells in the suspended state were immobilized using a 4% paraformaldehyde phosphate buffer, and analyzed using a flow cytometer in the same manner as above.

In the non-treated group, C3H10T1/2 cells were first seeded on a 12 well plate so that the cell density of the next day was 3.0×10⁵ cells/well, and cultured until the next day. Subsequently, a 15% FBS-containing DMEM medium was added in place of mitochondria, and the cells were cultured for 24 hours. After culturing, the cells were detached from the wells using a trypsin/EDTA solution (2.5 g/L trypsin, 1 mmol/L EDTA) and suspended in PBS. Then the cells in the suspended state were immobilized using a 4% paraformaldehyde phosphate buffer, and analyzed using a flow cytometer in the same manner as above.

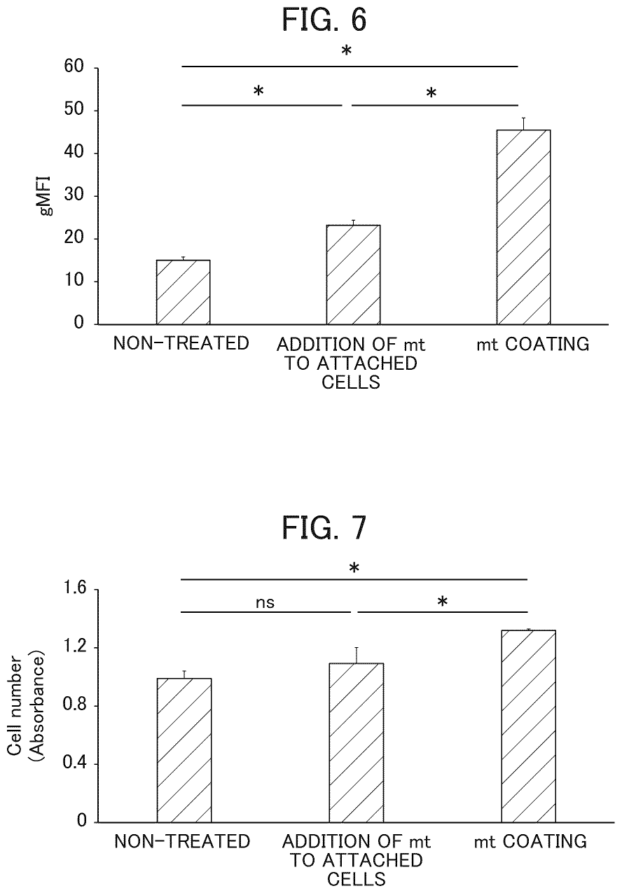

The geometric mean fluorescence intensity in each group is shown in FIG. 6. Each data in the graph shows the mean value±standard deviation of 3 samples. In addition, the “*” in the graph indicates that there is a statistical significance (*p<0.05; Tukey-Kramer's test). As can be seen from FIG. 6, when culturing the cells on a plate having the inside bottom surface of the wells coated with mitochondria, the amount of incorporated mitochondria was significantly increased compared to when not coating the inside bottom surface of the wells with mitochondria and adding mitochondria to the attached cells.

### Test Example 7: The Evaluation of the Proliferative Properties of Mitochondria-Introduced Cells

C3H10T1/2 cells (3.0×10⁵ cells/well) were seeded on a 12 well plate coated with mitochondria derived from C3H10T1/2 cells (3.0×10⁶ cells mt/well) and cultured for 24 hours to obtain cells having mitochondria introduced thereinto. For comparison, a group of adding mitochondria derived from C3H10T1/2 cells (3.0×10⁶ cells mt/well) to C3H10T1/2 cells in the attached state (3.0×10⁵ cells/well) was also prepared. For comparison, a non-treated group of using a 12 well plate not coated with mitochondria and adding a 15% FBS-containing DMEM medium in place of mitochondria was further prepared. Cells prepared by each method were seeded on a 96 well plate (Corning) at 5.0×10³ cells/well and cultured for 48 hours. Then the cell number after culturing was measured using Cell Counting Kit-8 (CCK-8) (FUJIFILM Wako Pure Chemical Corporation). Specifically, the CCK-8 solution was added at 100 μL/well and the obtained mixture was incubated for 30 minutes, and the absorbance at a wavelength of 450 nm was then measured to measure the cell number.

The relative value of the cell number in each group is shown in FIG. 7. Each data in the graph shows the mean value±standard deviation of 3 samples. In addition, the “ns” in the graph indicates that there is not a statistical significance, and the “*” indicates that there is a statistical significance (*p<0.05; Tukey-Kramer's test). As can be seen from FIG. 7, when culturing the cells on a plate having the inside bottom surface of the wells coated with mitochondria, the proliferative properties of the cells were significantly increased compared to when not coating the inside bottom surface of the wells with mitochondria and adding mitochondria to the attached cells.

### Test Example 8: The Evaluation of the Amount of ATP Production by Mitochondria-Introduced Cells

C3H10T1/2 cells (3.0×10⁵ cells/well) were seeded on a 12 well plate coated with mitochondria derived from C3H10T1/2 cells (3.0×10⁶ cells mt/well) and cultured for 24 hours to obtain cells having mitochondria introduced thereinto. For comparison, a non-treated group of using a 12 well plate not coated with mitochondria and adding a 15% FBS-containing DMEM medium in place of mitochondria was also prepared. After culturing, the cells were detached from the wells using a trypsin/EDTA solution (2.5 g/L trypsin, 1 mmol/L EDTA), and 100 μL of cell suspension and 100 μL of ATP assay reagent were mixed. The liquid after mixing was added to a 96 well plate (Corning) and allowed to stand for 10 minutes, and the luminescence amount derived from the cells was then measured using a microplate reader (EnVision, PerkinElmer).

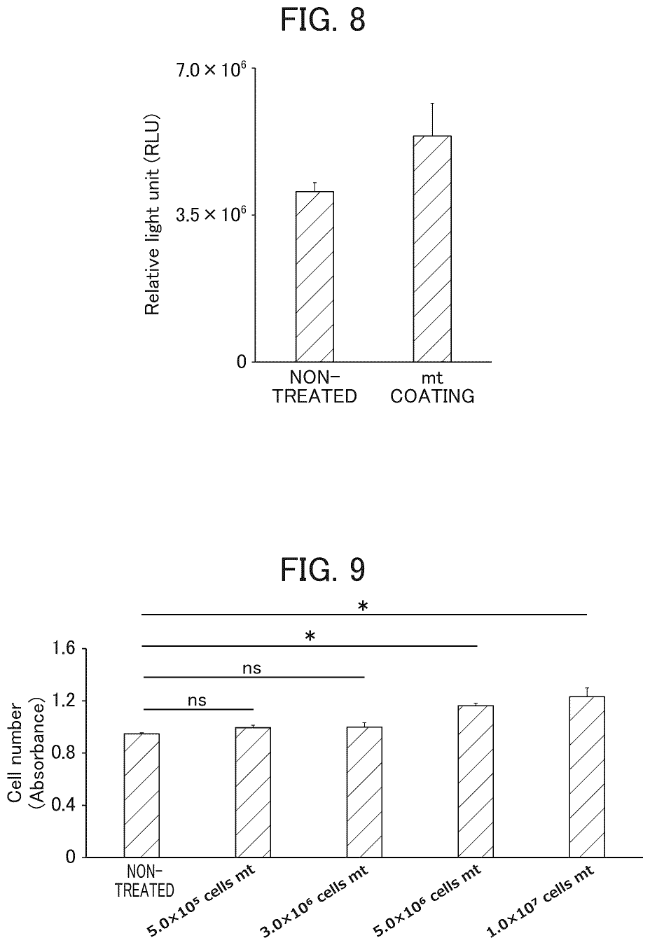

The luminescence amount in each group is shown in FIG. 8. Each data in the graph shows the mean value±standard deviation of 3 samples. As can be seen from FIG. 8, when culturing cells on a plate having the inside bottom surface of the wells coated with mitochondria, the amount of ATP production of the cells was largely increased.

### Test Example 9: The Evaluation of the Mitochondrial Number-Dependent Cell Proliferation

First, mitochondria were isolated from 2.0×10⁷ cells of C3H10T1/2 cells using the SLO method. The mitochondria were added to a 12 well plate at predetermined numbers (1.0×10⁵, 5.0×10⁵, 1.0×10⁶, 3.0×10⁶, 5.0×10⁶, 1.0×10⁷ cells mt/well), and plate centrifugation (1500 g, 10 minutes, 4° C.) was carried out to coat the inside bottom surface of the wells with the mitochondria. Subsequently, C3H10T1/2 cells were seeded at 3.0×10⁵ cells/well and cultured for 24 hours. After culturing, the cells were detached from the wells using a trypsin/EDTA solution (2.5 g/L trypsin, 1 mmol/L EDTA), seeded on a 96 well plate (Corning) at 1.0×10⁴ cells/well and cultured for 24 hours. Then the cell number after culturing was measured using a live cell assay kit (Cell Counting Kit-8 (CCK-8), FUJIFILM Wako Pure Chemical Corporation). Specifically, the CCK-8 solution was added at 100 μL/well and the obtained mixture was incubated for 30 minutes, and the absorbance at a wavelength of 450 nm was measured to measure the cell number.

For comparison, a non-treated group of the same operation as above except for using a 12 well plate not coated with mitochondria was also prepared.

The relative value of the cell number in each group is shown in FIG. 9. Each data in the graph shows the mean value±standard deviation of 3 samples. In addition, the “ns” in the graph indicates that there is not a statistical significance, and the “*” indicates that there is a statistical significance (*p<0.05; Dunnett's test). As can be seen from FIG. 9, the proliferative properties of the cells were increased in an introduced mitochondria number-dependent manner.

### Test Example 10: The Evaluation of the Proliferative Properties of Mitochondria-Introduced and Oligomycin-Treated Cells

First, C3H10T1/2 cells were seeded on a 6 well plate at 2.0×10⁵ cells/well. After 24 hours, the medium was replaced with a 15% FBS-containing DMEM medium containing 10 μg/mL oligomycin. After 24 hours, the cells were recovered using a trypsin/EDTA solution (2.5 g/L trypsin, 1 mmol/L EDTA) and used for the subsequent experiment as oligomycin-treated C3H10T1/2 cells.

Mitochondria were isolated from 3.0×10⁷ cells of C3H10T1/2 cells using the SLO method. The mitochondria were added to a 96 well plate (Corning) at predetermined numbers (1.0×10⁴, 5.0×10⁴, 1.0×10⁵, 3.0×10⁵, 5.0×10⁵, 1.0×10⁶ cells mt/well), and plate centrifugation (1500 g, 10 minutes, 4° C.) was carried out to coat the inside bottom surface of the wells with the mitochondria. Subsequently, oligomycin-treated C3H10T1/2 cells were seeded at 1.0×10⁴ cells/well and cultured for 24 hours. Then the cell number after culturing was measured using a live cell assay kit (Cell Counting Kit-8 (CCK-8), FUJIFILM Wako Pure Chemical Corporation). Specifically, the CCK-8 solution was added at 100 μL/well and the obtained mixture was incubated for 30 minutes, and the absorbance at a wavelength of 450 nm was then measured to measure the cell number.

For comparison, a non-treated group of the same operation as above except for using a 96 well plate not coated with mitochondria was also prepared.

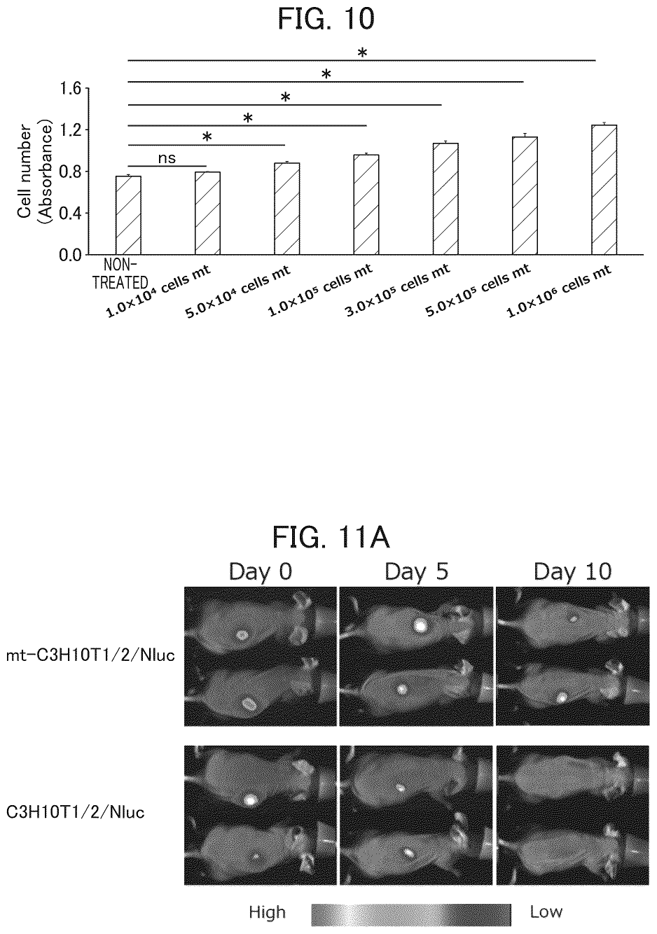

The relative value of the cell number in each group is shown in FIG. 10. Each data in the graph shows the mean value±standard deviation of 3 samples. In addition, the “ns” in the graph indicates that there is not a statistical significance, and the “*” indicates that there is a statistical significance (*p<0.05; Dunnett's test). As can be seen from FIG. 10, the proliferative properties of the cells were increased in an introduced mitochondria number-dependent manner also in the cells treated with oligomycin, a mitochondria inhibitor.

### Test Example 11: The Evaluation of Viability after Transplantation of Mitochondria-Introduced Cells

NanoLuc luciferase-expressing C3H10T1/2 cells (C3H10T1/2/Nluc cells) (3.0×10⁵ cells/well) were seeded on a 12 well plate coated with mitochondria derived from C3H10T1/2 cells (3.0×10⁶ cells mt/well) and cultured for 24 hours to obtain mitochondria-introduced C3H10T1/2/Nluc cells. The mitochondria-introduced C3H10T1/2/Nluc cells (mt-C3H10T1/2/Nluc) or non-treated C3H10T1/2/Nluc cells were prepared at 5.0×10⁵ cells (200 μL) and subcutaneously administered to the back of male BALB/c-nu/nu mice aged 6 weeks. Then 50 μL of luciferin (Nano-Glo, Promega) was administered to the cell-administered site, and in vivo imaging was carried out chronologically using an imaging device (In-Vivo Xtreme, Bruker Daltonik GmbH). In addition, blood was collected over time and plasma luciferase activity was measured using a microplate reader (EnVision, PerkinElmer).

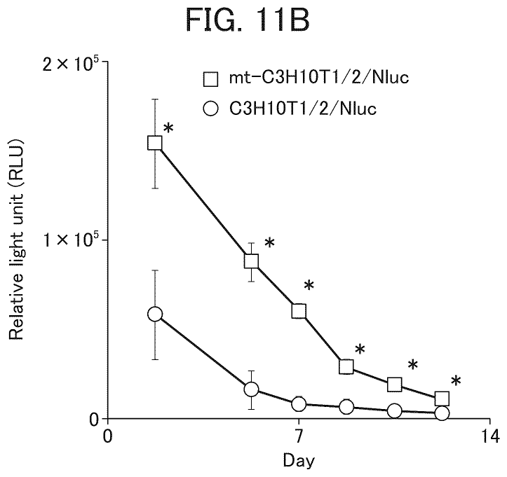

The in vivo images in each group are shown in FIG. 11A, and the plasma luciferase activity is shown in FIG. 11B. Each data in FIG. 11B shows the mean value±standard deviation of 3 samples. In addition, the “*” in FIG. 11B indicates that there is a statistical significance (*p<0.05; Dunnett's test). As can be seen from FIG. 11A and FIG. 11B, the cell viability after transplantation under the skin of mice was significantly raised by introducing mitochondria into C3H10T1/2/Nluc cells.

### Test Example 12: The Treatment Effect in Hepatopathy Model Mice

NanoLuc luciferase-expressing C3H10T1/2 cells (C3H10T1/2/Nluc cells) (3.0×10⁵ cells/well) were seeded on a 12 well plate coated with mitochondria derived from C3H10T1/2 cells (5.0×10⁶ cells mt/well) and cultured for 24 hours to obtain mitochondria-introduced C3H10T1/2/Nluc cells. Meanwhile, carbon tetrachloride (CC14) was intraperitoneally administered at 1 mL/kg body weight to male ddY mice aged 6 weeks to make a hepatopathy model mouse. At 6 hours after the administration of CC14, 8.0×10⁵ cells of mitochondria-introduced C3H10T1/2/Nluc cells (mt-C3H10T1/2/Nluc) or untreated C3H10T1/2/Nluc cells were intravenously administered, and blood was collected after 24 hours. Then serum AST and ALT values were measured using a commercially available kit (Transaminase C II-test Wako, FUJIFILM Wako Pure Chemical Corporation).

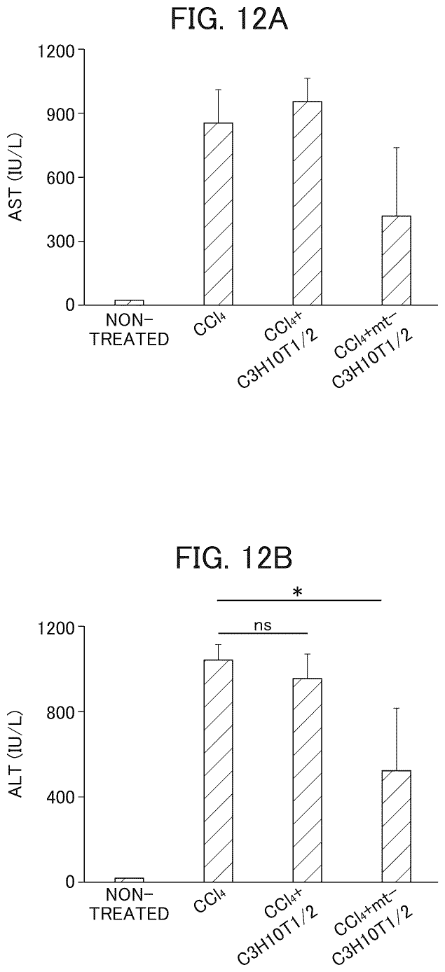

The serum AST value is shown in FIG. 12A and the serum ALT value is shown in FIG. 12B. Each data in the graphs shows the mean value±standard deviation of 3 samples. In FIG. 12B, the “ns” indicates that there is not a statistical significance, and the “*” indicates that there is a statistical significance (*p<0.05; Dunnett's test). As can be seen from FIG. 12A and FIG. 12B, a high treatment effect on the hepatopathy model mouse was shown by introducing mitochondria into C3H10T1/2/Nluc cells.

### Test Example 13: The Evaluation of the Proliferative Properties of Mitochondria-Introduced Cells

Mitochondria were isolated from 1.0×10⁸ cells of C3H10T1/2 cells using the SLO method. The mitochondria were added to a 96 well plate at 1 μg/well (50 μL/well), and plate centrifugation (1500 g, 10 minutes, 4° C.) was carried out to coat the inside bottom surface of the wells with the mitochondria. Subsequently, C3H10T1/2 cells were seeded at 1.0×10³ cells/well, Hepa1-6 cells (mouse hepatoma cell line) at 2.0×10³ cells/well, HEK293 cells (human embryotic kidney cell line) at 2.0×10³ cells/well, or HaCaT cells (human epidermal keratinocyte cell line) at 1.0×10³ cells/well, and cultured for 48 hours. Then the cell number after culturing was measured using a live cell assay kit (Cell Counting Kit-8 (CCK-8), FUJIFILM Wako Pure Chemical Corporation). Specifically, the CCK-8 solution was added at 100 μL/well and the obtained mixture was incubated for 30 minutes, and the absorbance at a wavelength of 450 nm was then measured to measure the cell number.

For comparison, a non-treated group of the same operation as above except for using a 96 well plate not coated with mitochondria was also prepared.

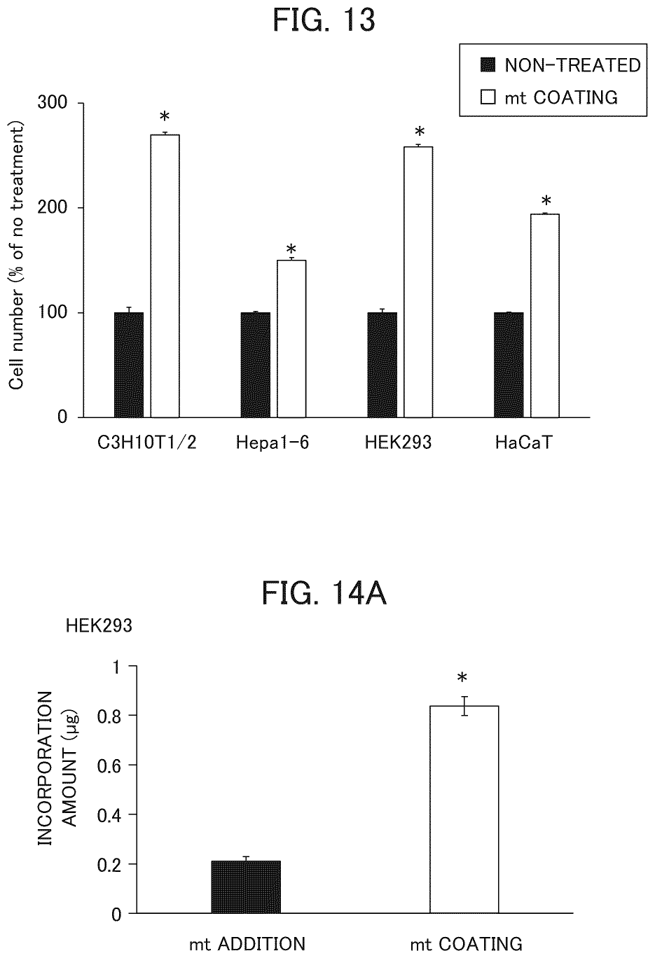

When the mean value of the cell number in the non-treated group is considered 100%, the relative value of the cell number is shown in FIG. 13. Each data in the graph shows the mean value±standard deviation of 3 samples. In addition, the “*” in the graph indicates that there is a statistical significance (*p<0.05; Student's t-test). As can be seen from FIG. 13, when any of C3H10T1/2 cells, Hepa1-6 cells, HEK293 cells and HaCaT cells was used as the recipient cells, the proliferative properties of cells were significantly increased.

### Test Example 14: The Comparison of the Amount of Incorporated Mitochondria

Mitochondria were isolated from 1.2×10⁸ cells of C3H10T1/2 cells using the SLO method, and the protein amount was measured using a protein assay kit (Pierce BCA Protein Assay Kits, Thermo Scientific). The mitochondria were added on a 12 well plate at 80 μg/well (500 μL/well), and plate centrifugation (1500 g, 10 minutes, 4° C.) was carried out to coat the inside bottom surface of the wells with the mitochondria. Subsequently, HEK293 cells or HaCaT cells were seeded at 2.0×10⁵ cells/well and cultured for 24 hours. After culturing, the cells were detached from the wells using a trypsin/EDTA solution (2.5 g/L trypsin, 1 mmol/L EDTA) and cellular RNA was collected using a total RNA purification kit (Monarch Total RNA Miniprep Kit, BioLabs). Subsequently, reverse transcription to cDNA was carried out using a reverse transcription reaction kit (ReverTra Ace qPCR RT Master Mix with gDNA Remover, TOYOBO Co., Ltd.) by a thermal cycler (GeneAtlas, ASTEC Co., Ltd.). The reverse transcription was sequentially carried out at 37° C. for 15 minutes, 50° C. for 5 minutes and 98° C. for 5 minutes. Subsequently, mouse COX2 gene was PCR-amplified using a real-time PCR reagent (THUNDERBIRD SYBR qPCR Mix, TOYOBO Co., Ltd.) by a real-time PCR analysis system (CFX Connect, Bio-Rad). As the protocol for the PCR, a cycle of 95° C. for 30 seconds, then 95° C. for 5 seconds, 55° C. for 15 seconds and 72° C. for 45 seconds was carried out for 39 cycles. In addition, the following primers were used for the PCR amplification of mouse COX2 gene.
Forward primer: 5′-CCATCCCAGGCCGACTAA-3′ (SEQ ID NO:1)
Reverse primer: 5′-AATTTCAGAGCATTGGCCATAGA-3′ (SEQ ID NO: 2)

Then a calibration curve was made about a relationship between the protein amount in mitochondria (μg) and the number of COX2 cycles (Cq value), and the protein amount in mitochondria incorporated into respective cells was calculated by substituting the number of COX2 cycles in the respective cells for the calibration curve.

For comparison, a group of the same operation as above except for not coating the inside bottom surface of the wells with mitochondria and adding mitochondria after seeding HEK293 cells or HaCaT cells was also prepared.

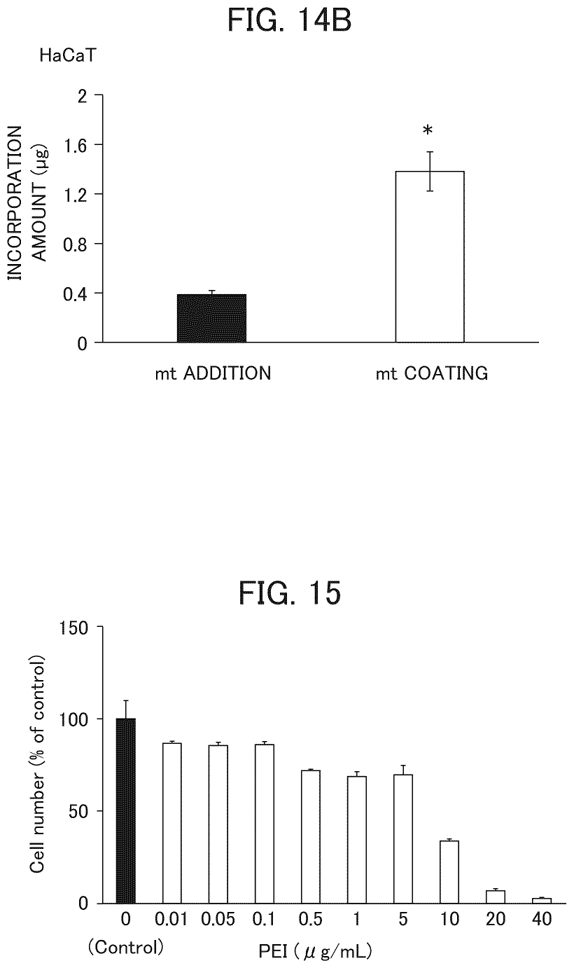

The protein amount in mitochondria incorporated into HEK293 cells is shown in FIG. 14A and the protein amount in mitochondria incorporated into HaCaT cells is shown in FIG. 14B. Each data in the graphs shows the mean value±standard deviation of 3 samples. In addition, the “*” in the graphs indicates that there is a statistical significance (*p<0.05; Student's t-test). As can be seen from FIG. 14A and FIG. 14B, the protein amounts in mitochondria incorporated per 2.0×10⁵ cells were about 0.8 μg in HEK293 cells and about 1.4 μg in HaCaT cells.

### Test Example 15: The Coating of a Plate for Producing a Cell Sheet with Mitochondria

First, mitochondria were isolated from 2.0×10⁷ cells of C3H10T1/2 cells using the SLO method. A 10 UM CFSE solution (1 mL) was added to the isolated mitochondria, and the obtained mixture was allowed to stand on ice for 30 minutes to stain the mitochondria. The stained mitochondria were washed with PBS twice and then suspended in a 15% FBS-containing DMEM medium (37° C.) (375 μL). Subsequently, the stained mitochondria were added to a 24 well plate for producing a cell sheet (Upcell, CellSeed Inc.) heated in advance to 37° C. at 2.0×10⁶ cells mt/well, and plate centrifugation (1500 g, 10 minutes, 37° C.) was carried out. After washing with a 15% FBS-containing DMEM medium (37° C.), the wells were observed using a digital fluorescence microscope (BZ-X800, KEYENCE CORPORATION). Consequently, mitochondria were observed to be attached on the inside bottom surface of the wells.

### Test Example 16: The Production of a Cell Sheet Using a Plate Coated with Mitochondria

First, mitochondria were isolated from 2.0×10⁷ cells of C3H10T1/2 cells using the SLO method and suspended in a 15% FBS-containing DMEM medium (37° C.) (375 μL). Subsequently, the stained mitochondria were added to a 24 well plate for producing a cell sheet (Upcell, CellSeed Inc.) heated in advance to 37° C. at 2.0×10⁶ cells mt/well, and plate centrifugation (1500 g, 10 minutes, 37° C.) was carried out to coat the inside bottom surface of the wells with the mitochondria. Subsequently, C3H10T1/2 cells suspended in a 15% FBS-containing DMEM medium (37° C.) were seeded at 4.5×10⁵ cells/well and cultured for 24 hours. After culturing, the medium was removed from the plate and a 15% FBS-containing DMEM medium (50 μL) was quickly added thereto. Subsequently, a supporting body (Cell Shifter, CellSeed Inc.) was put on the cell sheet using tweezers so that air bubbles were not included, and was allowed to stand at normal temperature (25° C.) for 5 minutes. Then the supporting body was peeled off from the plate and allowed to stand, and the 15% FBS-containing DMEM medium was then added dropwise so that the supporting body was immersed, and the supporting body was peeled off using tweezers. Consequently, the cell sheet having exogenous mitochondria introduced thereinto could be collected.

### Reference Example 1: The Cytotoxicity of a Cationic Substance

C3H10T1/2 cells were seeded on a 96 well plate at 5.0×10³ cells/well, polyethylenimine (PEI) diluted in PBS was then added thereto at various final concentrations of 0 to 40 μg/mL, and the obtained mixture was cultured for 24 hours. The used PEI was in a branched form, the mass average molecular weight thereof obtained by a light scattering method was about 25000 and the number average molecular weight thereof obtained by gel permeation chromatography was about 10000. Then the cell number after culturing was measured using a live cell assay kit (Cell Counting Kit-8 (CCK-8), FUJIFILM Wako Pure Chemical Corporation). Specifically, the CCK-8 solution was added at 100 μL/well and the obtained mixture was incubated for 30 minutes, and the absorbance at a wavelength of 450 nm was then measured to measure the cell number.

When the mean value of the cell number in a group of not adding PEI is considered 100%, the relative value of the cell number is shown in FIG. 16. Each data in the graph shows the mean value±standard deviation of 3 samples. As described in an existing report (Moghimi et al., Mol. Ther., 11:990-5 (2005)), PEI showed cytotoxicity at a concentration of 10 μg/mL or more.

What is claimed is:

1. A method of producing cells having exogenous mitochondria introduced thereinto, the production method comprising using cell culture equipment in which a culture surface is coated with mitochondria isolated from donor cells, attaching recipient cells to the culture surface, and culturing the recipient cells to make the recipient cells incorporate the mitochondria.

2. The production method according to claim 1, wherein the cell culture equipment is a cell culture vessel or a cell culture carrier.

3. (canceled)

4. (canceled)

5. Cell culture equipment, comprising a culture surface coated with isolated mitochondria.

6. The cell culture equipment according to claim 5, which is a cell culture vessel or a cell culture carrier.

7. A method of producing a cell culture vessel having a culture surface coated with isolated mitochondria, the production method comprising centrifuging the cell culture vessel with a liquid containing the isolated mitochondria added to the cell culture vessel to attach the mitochondria to the culture surface of the cell culture vessel.

8. (canceled)
